(19) United States
(12) Patent Application Publication
(10) Pub. No.: US 2026/0086102 A1
(43) Pub. Date: Mar. 26, 2026

(54) PROCESS OF PREPARING A MACROPOROUS HYDROGEL, AND IMPLEMENTATIONS THEREOF

(71) Applicant: ACHIRA LABS PVT. LTD., Bengaluru (IN)

(72) Inventors: Soumya BHATTACHARYA, Bengaluru (IN); Dhananjaya DENDUKURI, Bengaluru (IN)

(73) Assignee: ACHIRA LABS PVT. LTD., Bengaluru (IN)

(21) Appl. No.: 19/338,923

(22) Filed: Sep. 24, 2025

(30) Foreign Application Priority Data
Sep. 24, 2024 (IN) 202441072235

(51) Int. Cl. G01N 33/78; C08F 122/10; C08J 9/28; G01N 33/543

(52) U.S. Cl. CPC G01N 33/78; C08F 122/1006; C08J 9/286; G01N 33/54386; C08J 2201/0464; C08J 2205/022; C08J 2335/02; G01N 2333/59

(57) ABSTRACT

The present disclosure discloses a process for preparing a macroporous hydrogel using spinodal decomposition technique. The present disclosure provides a macroporous hydrogel and a macroporous hydrogel sensor. Further, the present disclosure provides a method of detecting an analyte in a sample using the macroporous hydrogel sensor, and a point of care kit comprising the macroporous hydrogel sensor.

### RELATED APPLICATION

The present application claims priority to Indian Patent Application No. 202441072235, filed Sep. 24, 2024, the entire contents of which are hereby incorporated by this reference.

### FIELD OF INVENTION

The present disclosure broadly relates to the field of hydrogels, and a method for preparation of macroporous hydrogels and sensors.

### BACKGROUND OF THE INVENTION

Polyethylene glycol (PEG)-based hydrogel structures hold tremendous promise in a variety of applications including biosensing, organ-on-chip and tissue engineering. When used as biosensors for immune and nucleotide-based assays (Le Goff G C et al. Hydrogel microparticles for biosensing. Eur Polym J. 2015 November; 72:386-412.), these porous platforms serve as micro-reactors for immobilizing antibodies or oligonucleotides which can be specifically bound to a target analyte (antigens/antibodies/cDNA), enabling their detection in very minute quantities (as low as pg-ng/mL). Hydrogel based networks formed from such PEG-based precursors are quite dense, achieving high reagent loading but pore sizes are quite small (<100 nm) compared to other porous sensing media like nitrocellulose (>1 μm). The optimization of hydrogel porosity and interconnectivity holds the key for achieving enhanced transport of analytes within the gel matrix that can enable better sensitivity calculated via the Lower Limit of Detection (LLOD).

Current strategies for pore formation in hydrogels include freeze drying, phase separation and the use of porogens. While phase separation is a simple process, current methods use organic solvents that may not be biocompatible and do not facilitate control over pore size that is required, especially the −1 μm pores needed for the free diffusion of large macromolecules. Current methods such as freeze drying involve multiple steps and are complex and not easy to reproduce. Further, the use of beads or large particles to increase pore size can result in large pore sizes, but with poor interconnectivity.

Coupled to this, their small size and enhanced structural integrity helps in automated handling in narrow confinements, thus, paving the way for large scale integration with microfluidic devices. One challenge seen with hydrogel-based immunoassays is the non-specific signal due to the trapping of large analytes (>200 kDa) within the gel matrix. This non-specific signal is due to the entropic trapping of biomolecules and deteriorates the LLOD because of the high background signal. Since, the transport is essentially diffusion dominated, higher pore size with better interconnectivity will ensure better permeability of biomolecules with reduced background signal.

### SUMMARY OF INVENTION

In an aspect of the present disclosure, there is provided a process for preparing a macroporous hydrogel, the process comprising: exposing a prepolymer solution comprising an ethylene glycol-based monomer, a photoinitiator and at least one porogen, to UV radiation for a duration in the range of 0.1 s to 600 s to form a hydrogel; and washing the hydrogel, to obtain the macroporous hydrogel; wherein power density of the UV radiation is in the range of 0.1 to 70 mW/cm².

In an aspect of the present disclosure, there is provided a macroporous hydrogel obtained by the process as disclosed herein.

In an aspect of the present disclosure, there is provided a macroporous hydrogel sensor obtained by the process as disclosed herein.

In an aspect of the present disclosure, there is provided a method for detecting an analyte in a sample, wherein said method comprises contacting the macroporous hydrogel obtained by the methods as described herein or the sensor as disclosed herein and the sample; and detecting the presence or absence of the analyte in the sample.

In an aspect of the present disclosure, there is provided a kit comprising the macroporous hydrogel obtained by the method as disclosed herein or the sensor as disclosed herein, and an instruction manual.

These and other features, aspects, and advantages of the present subject matter will be better understood with reference to the following description. This summary is provided to introduce a selection of concepts in a simplified form. This summary is not intended to identify key features or essential features of the disclosed subject matter, nor is it intended to be used to limit the scope of the disclosed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts a phase diagram of a liquid-liquid mixture showing the binodal and spinodal regions; (a) T vs X plot, where T is the temperature and X is the molar concentration of chemical species; (b) G (T) vs X plot, where G(T) is the Gibbs Potential and X is the molar concentration of chemical species; arrows indicating the regions likely to get spinodal decomposition (SD), and nucleation and growth (also referred to herein as “N&G”), in accordance with the embodiments herein.

FIG. 2 depicts an exemplary gel making device (100) and solar simulator setup (200); wherein (a) depicts top view of the gel making device (100), depicting cavity filled with prepolymer solution (“prepolymer mixture” or “PPM”) (102), plastic cover (104), 3M tape (106), and glass slide (108); and (b) is a schematic representation of the solar simulator (Abet technologies) setup (200) with the gel making device placed on the sample stage (206), the glass slide (108) faces the ultraviolet (UV) light source (202); the photomask (204) is placed on the glass slide with the neutral density (ND) filter (not shown) kept on top of the photomask, in accordance with the embodiments herein.

FIG. 3 depicts hexagonal gels synthesized, wherein (a), (b), (c), (d), (e), and (f) represent hydrogels syntehsized without ND filters and (g), (h) and (i) represent hydrogels synthesized with ND1 filters; Images acquired in bright field at 2×, 10× and 40× magnification, in accordance with the embodiments herein.

FIG. 4 depicts gels synthesized with an exposure time (Tₑₓₚ) of 25 minutes (≈1500 s): wherein (a) depicts irregular shaped gels and wherein (b) and (c) depicts formation of droplets within the gel matrix, in accordance with the embodiments herein.

FIG. 5 depicts optical images of irregular shaped hydrogel microparticles synthesized with stacked neutral density filters and varying exposure time (Tₑₓₚ) at 10, 30, 15 and 17.5 minutes, wherein (a) depicts image of irregular shaped hydrogel microparticles synthesized with neutral density filters ND 2.1 at Tₑₓₚ (10 mins); (b) depicts image of irregular shaped hydrogel microparticles synthesized with neutral density filters ND 2.3 at Tₑₓₚ (30 mins); (c) depicts image of irregular shaped hydrogel microparticles synthesized with neutral density filters ND 2.3 at Tₑₓₚ (15 mins); (d) depicts image of irregular shaped hydrogel microparticles synthesized with neutral density filters ND 2.3 at Tₑₓₚ (17.5 mins); (e) depicts image of irregular shaped hydrogel microparticles synthesized with neutral density filters ND 2.3 at Tₑₓₚ (30 mins); and (f) depicts image of irregular shaped hydrogel microparticles synthesized with neutral density filters ND 2.3 at Tₑₓₚ (17.5 mins); Images taken at 2× magnification in the phase contrast mode, in accordance with the embodiments herein.

FIG. 6 depicts confocal micrographs of gels at Tₑₓₚ of 8 s, 80 s and 270 s taken at different Z-planes along with the corresponding thresholded images in ImageJ; average pore size and porosity increases with increasing UV exposure time, in accordance with the embodiments herein.

FIG. 7 is a representation of pore size distribution of 8 s, 80 s and 270 s gels estimated from confocal microscopy, in accordance with the embodiments herein.

FIG. 8 depicts field emission-scanning electron microscopy (FE-SEM) micrographs of gels synthesized at different Tₑₓₚ; wherein (a) depicts 8 s gel (gel centre), (b) depicts 8 s gel (gel edge) and (c) depicts 80 s gel (gel centre) taken at 10000× magnification; (d) depicts 270 s gel (gel centre), (e) depicts 480 s gel (gel centre) and (f) depicts 660 s gel (gel centre) taken at 5000× magnification, in accordance with the embodiments herein.

FIG. 9 depicts a comparative plot showing the pore size distribution of 8 s, 80 s, 270 s, 480 and 660 s gels estimated from FE-SEM, in accordance with the embodiments herein.

FIG. 10 is the ANOVA test result representing the statistical difference between the average pore diameters (in nm) of hydrogels vs their UV exposure time obtained on analyzing the SEM images using ImageJ software, in accordance with the embodiments herein; no significant difference was observed between 8 s and 80 s gels (p>0.5), however, significant differences were observed between 8 s and 270 s (p<0.01 represented as **), 8 s and 480 s (p<0.01) and 8 s and 660 s (p<0.01). for p values less than 0.05 (marked as double stars for 0.01 and single star for 0.05).

FIG. 11 depicts comparative TSH immunoassay calibration curves of 8 s, 80 s, 270 s, and 480 s gels, in accordance with the embodiments herein.

FIG. 12 depicts the variation of assay signal for gels synthesized with different UV exposure times (8 s, 80 s, 270 s and 480 s); signals measured at varying thyroid stimulating hormone (TSH) concentration, in accordance with the embodiments herein.

FIG. 13 depicts fluorescence images (imaged in Leica DMi8 microscope) of 8 s and 80 s hydrogel microsensors undergoing TSH immunoassay with four calibrators (A, E, G and I), wherein (a) depicts 8 s gel (A calibrator) and (b) depicts 8 s gel (E calibrator); (c) depicts 80 s gel (A calibrator), (d) depicts 80 s gel (E calibrator); (e) depicts 8 s gel (G calibrator); (f) depicts 8 s (I calibrator); (g) depicts 80 s gel (G calibrator); and (h) depicts 80 s gel (I calibrator), in accordance with the embodiments herein.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

The term “including” is used to mean “including but not limited to”. “Including” and “including but not limited to” are used interchangeably.

The terms “% w/v,” as used herein, refers to percentage by weight, relative to the volume of the total composition, unless otherwise specified.

The term “hydrogel”, as used herein, refers to hydrophilic, three-dimensional, cross-linked polymeric network. Typically, it is prepared from synthetic or natural polymers using polymerization techniques, for example using photopolymerization. The terms “hydrogel” and “gel” are used interchangeably throughout the present disclosure.

The term “photopolymerization”, as used herein, refers to a process wherein a reactive group undergoes polymerization using light-generated radicals. Typically, photopolymerization involves a liquid solution transforming into a gel or solid upon exposure to light. The liquid solution is a curable formulation of monomers, oligomers, or viscous prepolymers, along with a photoinitiator, and the gel or solid is a cured material, which is generally a polymer or polymer network, such as a hydrogel. The light may include a wide range of wavelengths depending on the photoinitiator used and the desired application of the hydrogel.

The term “polymerization induced phase separation” or “PIPS” as used herein refers to separation of two or more substances in a mixture into distinct phases, and wherein such phase separation is induced by polymerization of one or more substances.

The term “photoinitiator”, as used herein, refers to a substance that absorb photons upon irradiation with light within a given spectral range, and forms reactive species out of excited state, which initiates polymerization. Examples of photoinitiators include, but are not limited to, 2-hydroxy-2-methyl-propiophenone, lithium phenyl-2,4,6-trimethylbenzoylphosphinate, or 2-hydroxy-4′-(2-hydroxyethoxy)-2-methylpropiophenone.

The term “spinodal decomposition” or “SD”, as used herein refers a mechanism where a single thermodynamic phase spontaneously separates out into two distinct phases, bypassing the nucleation and growth (N&G). Accordingly, the present disclosure exploits the physics of mixing and demixing of liquid-liquid mixtures triggered by changes in the Gibbs Free Energy (ΔG^{mix}) during the PIPS process. Thermodynamically, the Gibbs Free Energy of mixing that governs the phase separation of a multicomponent liquid-liquid mixture is given by the relation:

$$Δ {G}^{mix} = Δ {H}^{mix} - T Δ {S}^{mix} \qquad ( 1 )$$

where ΔH^{mix} is the change in enthalpy, ΔS^{mix} is the change in entropy and T is the absolute temperature. The value of ΔG^{mix} determines the limits of thermodynamic stability beyond which phase separation is triggered.

In addition to this, any polymerization process is governed by the following relation:

$$Δ {G}_{polymerization} = Δ {H}_{polymerization} - T Δ {S}_{polymerization} \qquad ( 2 )$$

When, the monomer converts into a polymer, there is a transition from a disordered to an ordered state which renders ΔS_{polymerization} as negative. In general, the sign of ΔG_{polymerization} depends on the balance between enthalpic and entropic contributions. However, for free radical polymerization of PEGDA, both ΔS_{polymerization} & ΔH_{polymerization} have negative values, where ΔS_{polymerization}<0 signify entropy loss and ΔH_{polymerization}<0 signify exothermic C═C bond formation. Under these conditions, the overall ΔG_{polymerization} remains negative, ensuring that polymerization proceeds spontaneously.

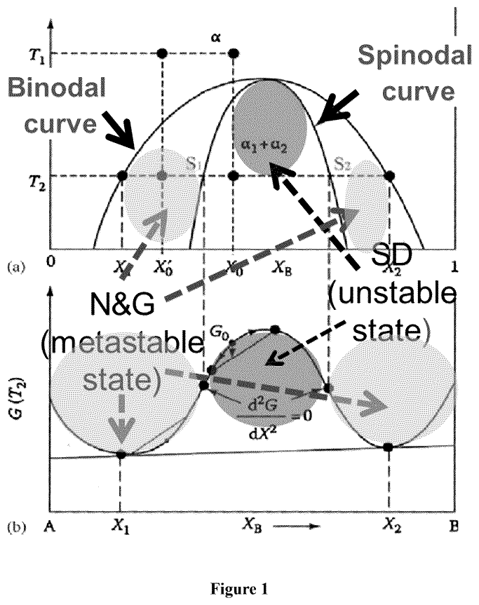

For PIPS to occur during the polymerization reaction, two possible phase separation mechanisms are involved: i) nucleation and growth (N&G) or ii) spinodal decomposition (SD). SD is a diffusion dominated process where a single thermodynamic phase spontaneously separates out into two distinct phases, a polymer rich phase and a polymer deficient one, bypassing the nucleation and growth kinetics. In the instant case (refer equation 1), the prepolymer solution is an unstable liquid-liquid mixture with a ΔG^{mix} value that falls within the blue shaded region within the spinodal curve in the phase diagram (FIG. 1); refer: “Phase Transformations in Metals and Alloys”, by D. A. Porter, K. E. Easterling and M. Y. Sherif, CRC Press, third edition) where the following relation holds true

$$\frac{{Δ}^{2} {G}^{mix}}{Δ {X}^{2}} < 0 \qquad ( 3 )$$

Spinodal decomposition inherently produces bi-continuous structures which if allowed, grow in size with time, capable of creating a highly interconnected porous polymeric network. This hydrodynamic growth driven by differences of surface tension between the polymer rich and polymer deficient phases dictates the microstructure of the resulting polymer network. The growth and coarsening of the bi-continuous structures are influenced by the fluctuation in local viscosity and diffusion rates of the components of the mixture.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in background, there are currently many challenges to be addressed in tuning porosity and interconnectivity in hydrogel-based structures for applications as sensors and in various assays, such as immunoassays. Conventional polymerization processes, like free radical polymerization, ionic gelation, chemical and physical crosslinking techniques for hydrogel synthesis rely more on nucleation and growth (N&G) kinetics for pore enhancement. Among several disadvantages of such conventional techniques, the major ones are: i) lack of control on the growth kinetics, ii) in-homogeneous pore size and distribution, iii) poor pore interconnectivity, iv) slow reaction kinetics leading to longer processing times and increased production costs, and iv) low throughput.

The present disclosure aims to address the issues of tuning porosity and pore interconnectivity in hydrogel structures by exploiting the phenomena of photopolymerization and spinodal decomposition whereby a single thermodynamic phase constituting a homogenous liquid-liquid mixture of a monomer and porogen solution spontaneously demixes or separates out into two phases by diffusive transport of chemical species without undergoing nucleation.

The present disclosure employs photo polymerization technique that induces crosslinking of monomer solution into hydrogel in presence of UV radiation. Similar to photolithography, the process is conducted within confined compartments filled with prepolymer solution and covered with photomasks. The photomasks have imprinted micro scale patterns that determine the size and shape of hydrogel microparticles. This enables one-pot synthesis of a plurality (for example in the order of hundreds or even thousands) of replicas of such micro-particle sensors, preferably with identical physico-chemical, mechanical and microstructural properties, in a fast and reproducible manner. This involves UV curing of prepolymer solution comprising the monomer and porogen in a flood UV-exposure system for high exposure times (of the order of minutes) and at very low light intensities (within 1-10 mW/cm²). This is brought about by placing Neutral Density optical filters over the photomasks that absorbs the UV light. The degree of UV-absorption depends on the optical density of the filters. The high exposure time coupled with low light intensity results in over 50% increase in mean pore size. The present disclosure highlights the pre-eminence of tuning the UV light intensity and exposure time for PIPS based large scale industrial manufacturing of hydrogel microparticles which along with a host of additional factors (such as the monomer concentration, monomer molecular weight, photoinitiator conc., porogen type and porogen concentration) complement in enabling substantial enhancement of pore size and interconnectivity.

The present disclosure achieves a process involving the use of ND filters to cut down the UV power intensity and increase the exposure time, that results in the slow baking of the prepolymer solution. Confocal and SEM characterization point to an increase in the average pore size from ≈200 to ≈460 nm with a much broader pore size distribution for an exposure time of 8 s to 80 s, 270 s and 480 s respectively.

Accordingly, the present disclosure provides a process for preparing macroporous hydrogel by inducing spinodal decomposition. The present disclosure provides a macroporous hydrogel and a macroporous hydrogel sensor. Further, the present disclosure provides a method of detecting an analyte in a sample using the macroporous hydrogel sensor, or a kit comprising the macroporous hydrogel sensor.

Embodiments herein provide a process for preparing a macroporous hydrogel. The term “macroporous” as used herein refers to materials having pore larger than 50 nm in size. Accordingly, the term “macroporous hydrogel”, as used herein refers to hydrogels that have porosities on both the nanoscale and the microscale.

In an embodiment of the present disclosure, there is provided a process for preparing a macroporous hydrogel, the process comprising: exposing a prepolymer solution comprising an ethylene glycol-based monomer, a photoinitiator and at least one porogen, to UV radiation for a duration in the range of 0.1 seconds to 1000 seconds or 0.1 seconds to 600 seconds, preferably 5 seconds to 500 seconds, more preferably 8 seconds to 480 seconds or 8 seconds to 270 seconds, to form a hydrogel having a polymeric network; and washing the hydrogel, to obtain the macroporous hydrogel.

In an embodiment of the present disclosure, there is provided a process for preparing a macroporous hydrogel, wherein exposing the prepolymer solution to UV radiation induces spinodal decomposition in the prepolymer solution.

In an embodiment of the present disclosure, there is provided a process for preparing a macroporous hydrogel, comprising: inducing spinodal decomposition in a prepolymer solution comprising an ethylene glycol-based monomer, a photoinitiator and at least one porogen, by exposing the prepolymer solution to UV radiation for a duration in the range of 0.1 seconds to 1000 seconds or 0.1 seconds to 600 seconds, preferably 5 seconds to 500 seconds, more preferably 8 seconds to 480 seconds or 8 seconds to 270 seconds, to form a hydrogel having a polymeric network; and washing the hydrogel, to obtain the macroporous hydrogel.

In an embodiment of the present disclosure, there is provided a process for preparing a macroporous hydrogel, the process comprising: exposing a prepolymer solution comprising an ethylene glycol-based monomer, a photoinitiator and at least one porogen, to UV radiation for a duration in the range of 0.1 seconds to 1000 seconds or 0.1 seconds to 600 seconds, preferably 5 seconds to 500 seconds, more preferably 8 seconds to 480 seconds or 8 seconds to 270 seconds, to form a hydrogel having a polymeric network; and washing the hydrogel, to obtain the macroporous hydrogel, wherein power density of the UV radiation is in the range of 0.1 to 70 mW/cm².

In an embodiment of the present disclosure, there is provided a process comprising exposing a prepolymer solution comprising an ethylene glycol-based monomer, a photoinitiator and at least one porogen, to UV radiation, wherein the ethylene glycol-based monomer and the porogen are in a weight ratio range of 1:50 to 50:1.

In an embodiment of the present disclosure, there is provided a process for preparing a macroporous hydrogel, the process comprising: inducing spinodal decomposition in a prepolymer solution comprising an ethylene glycol-based monomer, a photoinitiator and at least one porogen, by exposing the prepolymer solution to UV radiation for a duration in the range of 0.1 seconds to 1000 seconds or 0.1 seconds to 600 seconds, preferably 5 seconds to 500 seconds, more preferably 8 seconds to 480 seconds or 8 seconds to 270 seconds, to form a hydrogel having a polymeric network; and washing the hydrogel, to obtain the macroporous hydrogel; wherein power density of the UV radiation is in the range of 0.1 to 70 mW/cm²; and wherein the ethylene glycol-based monomer and the porogen are in a weight ratio range of 1:50 to 50:1. In an embodiment, the exposure time is selected from 8 s, 80 s, 270 s, or 480 s. In an embodiment, the porogen may be a combination of two or more porogens.

In an embodiment, the prepolymer solution comprises an ethylene glycol-based monomer, a photoinitiator and at least one porogen in an aqueous buffer.

The term “ethylene glycol-based monomer”, as used herein refers to monomeric substances comprising repeating units of ethylene glycol moieties, that are linked to become a chainlike polymer (solid) through a photopolymerization process. Further, to prevent the redissolving of these solid polymers into the liquid monomers, the polymers may be crosslinked adequately. To achieve polymer crosslinking, the ethylene glycol-based monomers are additionally modified with one or more functional groups selected from diacrylate, methacrylate, di-methacrylate, or norborene.

In an embodiment, the ethylene glycol-based monomer is selected from poly(ethylene glycol) di-acrylate (PEGDA), poly(ethylene glycol) methacrylate (PEGMA), poly(ethylene glycol) dimethacrylate (PEGDMA), PEG-norborene, or combinations thereof. In another embodiment, the ethylene glycol-based monomer is selected from poly(ethylene glycol) di-acrylate (PEGDA), poly(ethylene glycol) methacrylate (PEGMA), poly(ethylene glycol) dimethacrylate (PEGDMA), or combinations thereof. In yet another embodiment, the ethylene glycol-based monomer is poly(ethylene glycol) di-acrylate (PEGDA).

In an embodiment, the ethylene glycol-based monomer is PEGDA having an average molecular weight in the range of 250 to 6000 Da. In another embodiment, the ethylene glycol-based monomer is PEGDA having an average molecular weight in the range of 300 to 3000 Da. In yet another embodiment, the ethylene glycol-based monomer is PEGDA having an average molecular weight in the range of 600 to 1000 Da.

In an embodiment, the ethylene glycol-based monomer is in an amount in a range of 10 to 40% w/v with respect to the prepolymer solution. In yet another embodiment, the ethylene glycol-based monomer is in an amount in a range of 15 to 25% w/v with respect to the prepolymer solution.

In an embodiment, the ethylene glycol-based monomer is in an amount in a range of 15 to 25, 15 to 20, 16 to 19, 16.5 to 18, or 17 to 18.5% w/v with respect to the prepolymer solution.

In an embodiment, the ethylene glycol-based monomer is selected from poly(ethylene glycol) di-acrylate (PEGDA), poly(ethylene glycol) methacrylate (PEGMA), poly(ethylene glycol) dimethacrylate (PEGDMA), PEG-norborene, or combinations thereof; and the ethylene glycol-based monomer is in an amount in a range of 10 to 40% w/v with respect to the prepolymer solution.

In an embodiment, the photoinitiator is selected from 2-hydroxy-2-methyl-propiophenone, lithium phenyl-2,4,6-trimethylbenzoylphosphinate, 2-hydroxy-4′-(2-hydroxyethoxy)-2-methylpropiophenone, or combinations thereof. In another embodiment, the photoinitiator is 2-hydroxy-2-methyl-propiophenone.

In an embodiment, the photoinitiator is in an amount in a range of 0.1 to 2% w/v with respect to the prepolymer solution. In another embodiment, the photoinitiator is in an amount in a range of 0.2 to 2% w/v with respect to the prepolymer solution. In yet another embodiment, the photoinitiator is in an amount in a range of 0.3 to 1% w/v with respect to the prepolymer solution.

In an embodiment, the photoinitiator is in an amount in a range of 0.1 to 2, 0.15 to 2, 0.2 to 2, 0.25 to 2, 0.2 to 1, or 0.35 to 0.9% w/v with respect to the prepolymer solution.

In an embodiment, the photoinitiator is selected from 2-hydroxy-2-methyl-propiophenone, lithium phenyl-2,4,6-trimethylbenzoylphosphinate, 2-hydroxy-4′-(2-hydroxyethoxy)-2-methylpropiophenone, or combinations thereof; and the photoinitiator is in an amount in a range of 0.1 to 2% w/v with respect to the prepolymer solution.

The term “porogen” as used herein refers to substances that create, facilitate, or promote pore formation in polymeric networks. Typically, porogens have an influence on the pore size, pore morphology, pore interconnectivity, and porosity of the polymeric networks, such as hydrogels. Porogens, according to embodiments herein, may be an organic or inorganic compound, and either soluble or insoluble in water.

In an embodiment, the porogen is a water-soluble polymer or an inorganic compound.

In an embodiment, the water-soluble polymer is selected from polyethylene glycol (PEG), gelatin, agarose, alginate, or combinations thereof; and the inorganic compound is selected from sodium bicarbonate, silica, calcite, or combinations thereof.

In an embodiment, the porogen is a water-soluble polymer selected from PEG, gelatin, agarose, alginate, or combinations thereof. In a preferred embodiment, the water-soluble polymer is PEG.

In an embodiment, the water-soluble polymer is PEG having an average molecular weight in the range of 1 to 100 kDa. In another embodiment, the water-soluble polymer is PEG having an average molecular weight in the range of 1000 to 40 kDa. In yet another embodiment, the water-soluble polymer is PEG having an average molecular weight in the range of 10 kDa to 30 kDa.

In an embodiment, the porogen is in an amount in a range of 1 to 10% w/v with respect to the prepolymer solution. In an embodiment, the water-soluble polymer is in an amount in a range of 2 to 6% w/v with respect to prepolymer solution. In another embodiment, the water-soluble polymer is in an amount in a range of 3 to 6% w/v with respect to prepolymer solution.

In another embodiment, the porogen is an inorganic compound selected from sodium bicarbonate, silica, calcite, or combinations thereof. In a preferred embodiment, the inorganic compound is sodium bicarbonate. In an embodiment, the inorganic compound is in an amount in a range of 2 to 6% w/v with respect to the prepolymer solution. In another embodiment, the inorganic compound is in an amount in a range of 3 to 6% w/v with respect to the prepolymer solution.

In an embodiment, the water-soluble polymer is in an amount in a range of 1 to 10% w/v with respect to prepolymer solution; or an inorganic compound which is in an amount in a range of 1 to 10% w/v with respect to the prepolymer solution. In an embodiment, the porogen comprises the inorganic compound and water-soluble polymer in a weight ratio of 2:1 to 1:2, preferably 1:1. In an embodiment, the at least one porogen is in an amount in a range of 1 to 10% w/v with respect to prepolymer solution.

In an embodiment, the ethylene glycol-based monomer and the porogen are in a weight ratio range of 1:50 to 50:1, 10:1 to 1:10, or 5:1 to 1:1.

In an embodiment, the prepolymer solution comprises an aqueous buffer selected from phosphate buffered saline (PBS), tris-buffered saline (TBS) or combinations thereof. In another embodiment, the aqueous buffer is phosphate buffered saline (PBS).

In an embodiment, the process comprises: exposing a prepolymer solution comprising an ethylene glycol-based monomer, a photoinitiator and a porogen in an aqueous buffer, to UV radiation for a duration in the range of 0.1 s to 1000 s to form a hydrogel having a polymeric network; and washing the hydrogel, to obtain the macroporous hydrogel; wherein power density of the UV radiation is in the range of 0.1 to 70 mW/cm²; and wherein the ethylene glycol-based monomer and the porogen are in a weight ratio range of 1:50 to 50:1.

In an embodiment, the process comprises: inducing spinodal decomposition in a prepolymer solution comprising an ethylene glycol-based monomer, a photoinitiator and a porogen in an aqueous buffer, by exposing to UV radiation for a duration in the range of 0.1 s to 1000 s to form a hydrogel having a polymeric network; and washing the hydrogel, to obtain the macroporous hydrogel; wherein power density of the UV radiation is in the range of 0.1 to 70 mW/cm²; and wherein the ethylene glycol-based monomer and the porogen are in a weight ratio range of 1:50 to 50:1.

In an embodiment, the UV radiation passes through a photomask and one or more neutral density filters, wherein the neutral density filters have optical density in a range of 0.1 to 2 or 0.1 to 1.8.

The term “photomask”, as used herein refers to a solid, transparent substrate or film which has an opaque coating on one surface into which a microscopic pattern has been etched, leaving some regions transparent and others opaque. The opaque regions prevent/mask light from passing through, while the transparent regions allow light to pass through. Depending on the size, shape and the density of patterns/features printed on the photomasks, the light attenuation will vary (smaller feature sizes closely packed will attenuate light less than features with greater separation distance).

In an embodiment, the photomask is selected from laser-plotted polyester-based photomask, glass-based chrome films, or quartz-based chrome films having patterns on its surface of a size in the range of 50 to 500 microns. In embodiment, the photomask is selected from laser-plotted polyester-based photomask, glass-based chrome films, or quartz-based chrome films with micro scale patterned surfaces; and wherein the patterns have a size in the range of 50 to 500 microns. Examples of photomasks that may be used in the present disclosure include film photomasks comprising of polyester-based photoplot films and/or chrome photomasks.

The term “neutral density filter”, as used herein refers to filters crafted to uniformly decrease transmission within a designated spectrum range. These filters are commonly characterized by their Optical Density (OD), quantifying the extent of energy obstructed.

$$T ( Percent Transmission ) = {10}^{- OD} \times 100$$

A greater optical density signifies minimal transmission, while a lower optical density implies heightened transmission. For example, a ND2 filter with an OD of 2 allows minimum transmission of light, whereas ND1 filter with an OD of 1 allows higher transmission of light than ND1 filter. Custom optical densities can be attained by stacking ND filters. To determine the final OD of a configuration, one can sum the OD values of each individual filter. For example, ND1, ND0.3, and ND0.5 may be stacked together to obtain a cumulative effect similar to a single ND1.8 filter (OD 1.8=1+0.5+0.3).

In an embodiment, the neutral density filters have optical density in a range of 0.1 to 2, 0.1 to 1.8, or 0.5 to 2. In another embodiment, the neutral density filters have optical density in a range of 0.3 to 1.8. In yet another embodiment, the neutral density filters have optical density in a range of 0.5 to 1.8 or 1 to 1.8.

In an embodiment, spectral distribution of the radiation is in a range of 200 to 600 nm, and with a power density in the range of 0.1 to 70 mW/cm², preferably 0.1 to 65 mW/cm². In an embodiment, the power density is in the range of 0.1 to 5 mW/cm².

In an embodiment, washing is performed to remove unreacted ethylene glycol-based monomer and porogen. According to embodiments herein, washing the hydrogel is performed using a solvent like PBST (PBS with tween 20 buffer) under vortexing. In some embodiments, washing is performed multiple times to remove the unreacted ethylene-glycol based monomers, and porogens to obtain the macroporous hydrogel.

In an embodiment, there is provided a process for preparing a macroporous hydrogel, the process comprising: inducing spinodal decomposition in a prepolymer solution comprising an ethylene glycol-based monomer, a photoinitiator and a porogen, by exposing the prepolymer solution to UV radiation for a duration in the range of 0.1 s to 1000 s to form a polymeric network; and washing, to obtain the macroporous hydrogel; wherein power density of the radiation is in the range of 0.1 to 70 mW/cm²; and wherein the ethylene glycol-based monomer and the porogen are in a weight ratio range of 1:50 to 50:1; wherein the UV light passes through a photomask, and one or more neutral density filters, wherein the neutral density filters have optical density in a range of 0.1 to 1.8; wherein spectral distribution of the radiation is in a range of 200 to 600 nm, and with a power density in the range of 0.1 to 70 mW/cm²; wherein the photomask is selected from laser-plotted polyester-based photomask, glass based chrome films, or quartz-based chrome films with micro scale patterned surfaces; and wherein the patterns have a size in the range of 50 to 500 microns; and wherein washing is performed using a solvent comprising buffer, preferably PBS buffer, and surfactant, preferably Tween, to remove unreacted ethylene glycol-based monomer and porogen.

In an embodiment, there is provided a process for preparing a macroporous hydrogel, wherein the process comprises contacting the macroporous hydrogel with at least one capture agent and incubating to enable functionalization of the capture agent on the macroporous hydrogel to obtain a macroporous hydrogel sensor. In an embodiment, the capture agent is selected from an antibody, an oligonucleotide, an enzyme, an antigen, or combinations thereof. In an embodiment, the capture agent is a labelled captured agent.

In an embodiment, there is provided a process for preparing a macroporous hydrogel, wherein the process comprises lyophilizing said macroporous hydrogel to obtain a lyophilized macroporous hydrogel.

Embodiments herein provide a macroporous hydrogel. In an embodiment there is provided a macroporous hydrogel obtained by the process disclosed herein. In an embodiment, the macroporous hydrogel comprises pores having an average pore size in a range of 100 to 500 nm; and porosity in a range of 10 to 40%, preferably 15 to 38%.

Embodiments herein provide a macroporous hydrogel sensor.

In an embodiment, the macroporous hydrogel sensor is obtained by contacting the macroporous hydrogel obtained by the process as disclosed herein with at least one capture agent and incubating to enable functionalization of the capture agent on the gel to obtain the macroporous hydrogel sensor.

The term “capture agent”, as used herein refers to a substance that is capable of selectively binding to an analyte in a sample. Examples of capture agent include, but are not limited to, antibody, an oligonucleotide, an enzyme, or an antigen.

In an embodiment, the capture agent is selected from an antibody, an oligonucleotide, an enzyme, an antigen, or combinations thereof.

Embodiments herein provide a method for detecting an analyte in a sample.

In an embodiment, the method comprises contacting the macroporous hydrogel obtained by the methods as described herein or the macroporous hydrogel sensor as disclosed herein and the sample and detecting the presence or absence of the analyte in the sample. In an embodiment, the method comprises contacting the macroporous hydrogel obtained by the methods as described herein or the macroporous hydrogel sensor as disclosed herein and the sample and detecting the presence or absence of analyte in the sample by determining the presence or absence of analyte bound to the macroporous hydrogel sensor. In an embodiment, the presence or absence of analyte bound to the macroporous hydrogel sensor is determined by fluorometric detection. In an embodiment, the presence of analyte bound to the macroporous hydrogel sensor indicates the presence of the analyte in the sample, and absence of analyte bound to the macroporous hydrogel sensor indicates the absence of the analyte in the sample.

In an embodiment, the method comprises contacting the macroporous hydrogel obtained by the methods as described herein or the macroporous hydrogel sensor as disclosed herein and the sample along with a detection agent, and detecting the presence or absence of analyte in the sample. In an embodiment, detection of the analyte may be by fluorescence detection methods. Detection of the analyte by fluorescence labelling may be performed using a fluorophore tagged antibody, which then may bind to the analyte such as TSH. When excited by light, the fluorophore may emit a detectable fluorescent signal, allowing for the sensitive and specific visualization or quantification of the analyte using techniques such as fluorescence microscopy, flow cytometry, or immunoassays. This method provides specificity through antibody-antigen binding and allows for multiplexing by using multiple fluorescently labelled antibodies.

The term “detection agent” as used herein refers to an antibody or any other macromolecule that specifically binds to the analyte and enables detection of the analyte.

In an embodiment, the detecting agent comprises a label, and is capable of detecting the presence/absence of the analyte in the sample.

The term “label” as used herein refers to a molecule capable of enabling detection of other substances/molecules that are in association with the label. The label may be a fluorescent label, a chemiluminescent label, an enzyme label or a radio label. Examples of fluorescent labels include but are not limited to green fluorescent protein (GFP), mCherry, red fluorescent protein (RFP), yellow fluorescent protein (YFP), or cyan fluorescent protein (CFP). Examples of chemiluminescent labels include, but are not limited to, luminol and its derivatives, Acridinium esters, Peroxyoxalates, Dioxetanes, Lucigenin and Tris(2,2′-bipyridyl)ruthenium(II). Examples of enzyme label include, but are not limited to, Horseradish Peroxidase (HRP), Alkaline Phosphatase (AP), β-Galactosidase, Glucose Oxidase, Acetylcholinesterase (AChE), Creatinase and Urease. Examples of radiolabel include but are not limited to Iodine-125 (I-125), Tritium (H-3), Carbon-14 (C-14), Phosphorus-32 (P-32) and Sulfur-35 (S-35).

In an embodiment, the label is a fluorescent label or a chemiluminescent label. In another embodiment, the label is a fluorescent label.

In an embodiment, the analyte is selected from hormones (for e.g.: TSH, FSH), immunoglobulins (for e.g.: IgG), or antigenic proteins.

In an embodiment, the sample is obtained from a subject. In another embodiment, the sample is selected from blood, serum, plasma, urine, cerebrospinal fluid, or saliva obtained from a subject.

The term “subject”, as used herein, refers to mammals, including human and non-human mammals. Examples of non-human animals include non-human primates, dogs, cats, cattle, horses, sheep, pigs, goats, rabbits, mice, rats, hamsters, guinea pigs and etc.

Embodiments herein provide a kit. In an embodiment there is provided a kit comprising the macroporous hydrogel sensor obtained by the method as disclosed herein or the sensor as disclosed herein. In another embodiment there is provided a kit comprising the macroporous hydrogel sensor obtained by the method as disclosed herein or the sensor as disclosed herein, a microfluidic cartridge, a reader, and an instruction manual.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with following examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Materials

The following reagents and chemicals used in the present disclosure were procured from Sigma, Merck, Medix Biochemica, Biosynth, Biopharma-PEG & Dyomics.

PEG-diacrylate (PEGDA) Polyethylene glycol (PEG): PEGDA (Average Mol. Wt=700 Da, Product No. 455008, Sigma) and PEG (Mol. Wt=20000 Da), Product no. 81300, Sigma Aldrich; Sodium bicarbonate (NaHCO₃): NaHCO₃ was procured from Sigma, Product No. S7277; 2-hydroxy-2-methyl-propiophenone was procured from Sigma, Product No. 405655; PBS buffer: procured from Sigma, Product No. P4417; Tween 20: procured from Merck, Product No. 822184; reagents and chemicals for TSH assay: TSH, Thyrotropic Hormone from Human, Merck T9265, capture antibody: Anti-h TSH 5409 SPTNE-5 Medix Biochemica detection antibody: TSH beta antibody Biosynth (Cat. No.-T-25C); thiol-PEG-NHS linker (Cat. No. HE003023-2K), Biopharma PEG; Fluorescent dye, DY-647P4-NHS-Ester, Dyomics.

### Statistical Methods

All quantitative measurements were performed in triplicates as mean±standard deviation. Statistical significances were determined on both confocal and FE-SEM microscopy results using a one-way ANOVA test, and differences were considered significant for p values less than 0.05.

### Example 1

### Preparation and Characterization of Macroporous Hydrogel

### 1. Preparation of Macroporous Hydrogel

### Preparation of the Pre-Polymer Solution:

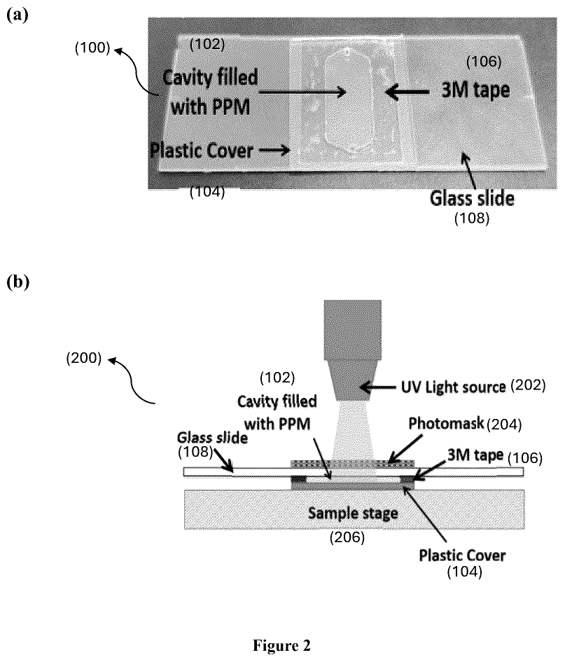

A prepolymer solution comprising measured volumes of PEGDA (MW=700 Da; 19% w/v) (a ethylene glycol-based monomer) mixed with 2-hydroxy-2-methyl-propiophenone (0.43% w/v) (a photoinitiator with strong absorption bands around 245-255 nm & 320-330 nm), PEG (MW=20 kDa; 5% w/v), and NaHCO₃ (5% w/v) were prepared in PBS buffer. NaHCO₃ and PEG both serve as porogens, wherein, NaHCO₃ (inorganic compound) is a soluble salt and PEG is a water soluble polymer. 40% w/v of PEG stock solution was prepared from which measured volumes were used as a liquid porogen. NaHCO₃ precipitates out and PEG along with water gets phase separated during spinodal decomposition, through photo-polymerization induced phase separation (PIPS). The PEG molecules do not take part in the polymerization process and undergo aggregation owing to their bulky size, eventually getting trapped within the polymer network of the hydrogel. Measured volumes (25 μL) of the prepolymer solution were filled in cavities in specially designed sealed devices (depicted in (a), FIG. 2) which were covered with film photo-masks and exposed to UV light (depicted in (b), FIG. 2). FIG. 2 depicts an exemplary gel making device (100) and solar simulator setup (200); wherein (a) depicts top view of the gel making device, depicting cavity filled prepolymer solution (102) (also referred to as “prepolymer mixture” or “PPM”), plastic cover (104), 3M tape (106), and glass slide (108); and (b) is a schematic representation of the solar simulator (Abet technologies) setup (200) with the gel making device placed on the sample stage (206), the glass slide (108) faces the ultraviolet (UV) light source (202); the photomask (204) was placed on the glass slide (108) with the neutral density (ND) filter (not shown) kept on top of the photomask, in accordance with the embodiments herein. The UV light reacts with the photo initiator to form free radicals that break the carbon-carbon double bonds of acrylate groups in PEGDA and join adjacent PEGDA chains through covalent linkage. The polymerization proceeds symmetrically via a chain growth type of mechanism leaving behind a 3D interconnected network of polymer chains. Post synthesis, the gel devices were opened by peeling off the plastic cover manually and the microgels were harvested and stored in PBST (PBS+0.1% Tween 20). Thereafter, they were washed with PBST with vortexing at 1200 rpm for 2 min. The washing step was repeated 3 times to flush out the unreacted PEGDA, NaHCO₃ and PEG, leaving behind a 3D porous micro-architechture.

### Preparation of the Hydrogel Using Solar Stimulator:

A standard flood exposure system (Abet solar simulator, refer FIG. 2, (b)) with 200 watt Hg arc lamp (OSRAM 200W/2 L1) was used as the light source (radiation) with a wide spectral distribution ranging from 200-600 nm. Neutral Density (ND) filters were crafted to uniformly decrease transmission within a designated spectrum range. The filters were stacked to achieve cumulative ODs. To determine the final OD of a configuration, the OD values of each individual filter were summed up. The light intensity was measured with an optical power meter PM100A (ThorLabs) kept below the photomask (laser-plotted polyester-based photomask having a size 500 micron) with ND filter placed on top of the mask (FIG. 2b). The light attenuation varies depending on the size, shape and the density of features printed on the photomasks (smaller closely packed feature sizes will attenuate light less than features with greater separation distance). The light intensity corresponding to various ND filter configurations and other details of exemplary embodiments are given in the following Table 1.1 and 1.2. The power density was in the range of 0.1 to 70 mW/cm².

TABLE 1.1
Examples of Prepolymer solution:

| | | | | Ratio of | | |
|---|---|---|---|---|---|---|
| | | | | ethylene | | |
| | Ethylene | | Porogen | glycol- | | |
| | glycol- | | (% w/v) | based | | |
| | based | | (Peg20 | monomer | PBS | |
| | monomer | Photoinitiator | kDa + | and the | Buffer | Total |
| Description | (% w/v) | (% w/v) | NaHCO3) | porogen | (% w/v) | (% w/v) |
| Prepolymer mix | 19 | 0.43 | 10 (5 + 5) | 1.9:1 | 70.57 | 100 |
| (WE1, WE2, WE3) | | | | | | |
| Prepolymer mix | 28 | 0.43 | 10 (5 + 5) | 2.8:1 | 61.57 | 100 |
| (WE4) | | | | | | |

TABLE 1.2
Light intensity values of Abet solar simulator

| | | Light | Power | UV |
|---|---|---|---|---|
| | ND Filter | Intensity | Density | Exposure |
| Description | configurations | (mW) | (mW/cm²) | time (Tₘ) |
| WE1 | Without ND | 49.1 | 62 | 8 s |
| | filter and | (max. | | |
| | photomask | value) | | |
| WE2 | ND1 (OD = 1) | 1.5 | 1.89 | 80 s |
| WE3 | ND 1.5 | 0.56 | 0.71 | 270 s |
| | (OD = 1 + 0.5) | | | |
| WE4 | ND 1.8 | 0.16 | 0.2 | 480 s |
| | (OD = 1 + 0.5 + 0.3) | | | |
| NWE6 | ND 2 (OD = 2) | 0.14 | 0.18 | 660 s |

### Preparation of the Hydrogel Using Fluorescence Microscope:

The macroporous hydrogel particles were also synthesized in Leica DMi8 inverted microscope to serve as a control. The gel device was filled with the prepolymer solution and exposed to the UV beam generated by an LED light source. Contrary to mercury arc lamps (in ABET) that emit a wide spectrum (covering UV-A &, UV-C wavelength ranges), the LED emits a narrow band of wavelength of 365 nm. Excitation wavelength of 365 nm was chosen at 100% intensity and 450 ms of exposure. An ROI was selected and the gels were produced one at a time controlled by the movable sample stage. Polymerization was confined to the exposure area guided by movable stage. The gel size depends on the mask size and the beam diameter produced by the UV LED source which passes through a photomask integrated in the optical filter cube of the microscope. The power density attained with such a narrow UV beam emitted by the light emitting diode (LED) was much higher (around 80 mW/cm²) than in case of Abet (62 mW/cm²).

### Characterization of Macroporous Hydrogels.

The macroporous hydrogels as prepared herein above were characterized using various microscopic techniques.

### Optical Microscopy:

After harvesting, the hydrogels were imaged in Nikon Eclipse Ti inverted microscope. Images were acquired in the bright field mode at 4×, 10×, 20× and 40× magnification.

### Confocal Microscopy:

### Dye Staining:

Dye staining of PEGDA hydrogels is a prerequisite for detecting pores in confocal microscopy. Normal incubation of gels in dye solution leads to surface adsorption and entrapment of the dye molecules in the polymer network. However, there is a high possibility that the dye molecules break loose and get washed away during washing steps. This leads to a very poor signal to noise ratio which affects image acquisition and analysis. Co-polymerization offers a viable alternative whereby the dye remains anchored strongly to the polymer network, thereby reducing chances of washout. 1% w/v stock solution of fluorescein-o-methacrylate (a fluorescent monomer) was prepared in PBS. The solubility of fluorescein-o-methacrylate in PBS being poor, the dye was vigorously vortexed for 1-2 min to get a uniform solution. Thereafter, it was added to the prepolymer solution and then photo polymerized. The final concentration of the dye was 0.1% w/v. Five different gels were prepared at different UV exposure times in Abet. The photoinitiator (PI) concentration was also varied; 8 s, 80 s and 270 s gels had 0.432% w/v PI, whereas, 480 s and 660 s gels had 0.864% w/v of PI; PEGDA and porogen concentration was same for all gels. All gels, except the 8 s ones, were synthesized using ND filters. Post synthesis, the dye-stained gels were harvested, washed and stored in PBS-0.1% (v/v) Tween buffer and harvested. To confirm the successful tagging of the dye, the gels were put on glass slides and imaged in Leica DMi8 fluorescence microscope with a blue-green filter (excitation=488 nm, emission=520 nm) at 10× magnification, 10% illumination and 100 ms exposure. A bright field image corresponding to the fluorescent one was also taken for accurate estimation of size and shape. Once the staining was confirmed, the gels were subsequently imaged in a confocal microscope.

### Confocal Imaging:

Images were procured using a 63× oil immersion objective at 3× magnification in Leica SP8 confocal laser scanning microscope (CLSM), after the gels were put on a glass coverslips and sandwiched with another coverslip after which it was fixed on the sample stage. An excitation wavelength of 488 nm was used, and the emission was measured at 520 nm; the numerical aperture was set to 1.4, pinhole was set at 107.9 μm corresponding to 1.13 pinhole airy units (AU). The raw images acquired were analysed using LASX software and a fluorescence image for a particular Z-value was chosen with the maximum signal to noise ratio, followed by contrast enhancement and digital magnification (300%). The pixel density of the raw image was 1024×1024 pixel² corresponding to a size of 61.51×61.51 micron², which decreased to 473×442 pixel² after digital magnification, keeping the scale factor unaltered (1 pixel=0.06 micron). The processed images were further analysed using ImageJ (https://www.academia.edu/24152953/Porosity_Analysis_Procedure_with_ImageJ; https://imagej.net/ij/docs/guide/146-30.html#toc-Section-30). The porosity (%) is the area in percentage estimated from ImageJ analysis.

For 8 s gels, a minimum void area of 0.018 μm² was chosen corresponding to a pore diameter of 151 nm and a maximum of 0.534 μm² corresponding to a pore diameter of 824 nm. This was done owing to a wide distribution of pore sizes in the sample, with an average of ˜0.059 μm² corresponding to a pore diameter of −324 nm. Void areas with values <0.0113 were ignored since they were closer to the lower limit of spatial resolution (≈120 nm) that could be imaged with the applied settings. Pore interconnectivity could not be ascertained with this technique. Similar process was applied for all other gel samples.

### Results

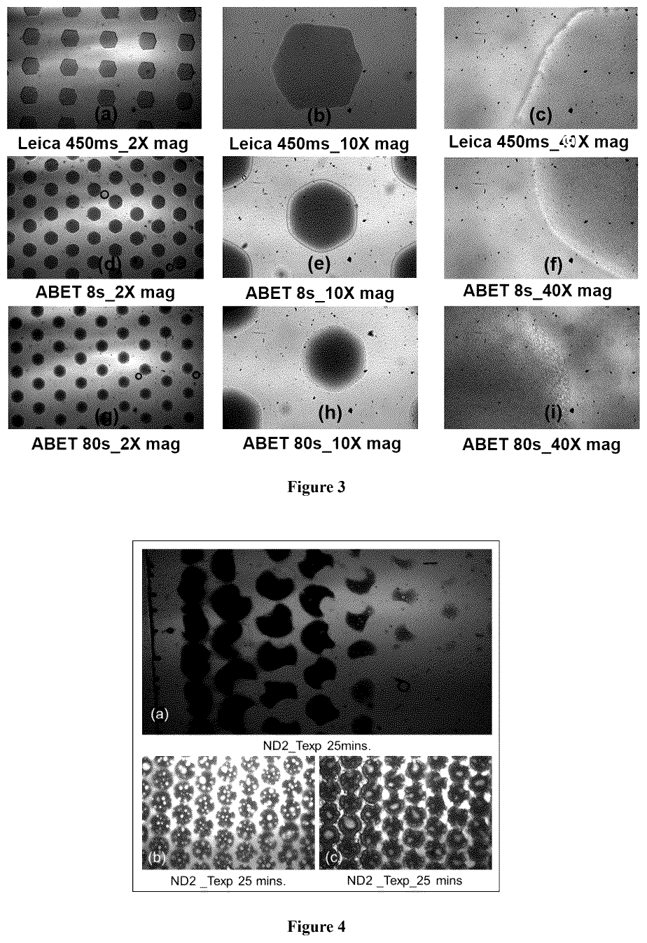

FIG. 3 depicts hexagonal gels synthesized, wherein (a), (b) and (c) and (d), (e), and (f) represents hydrogels syntehsized without ND filters and (g), (h) and (i) represents hydrogels syntehsized with ND1 filters; Images acquired in bright field at 2×, 10× and 40× magnification, in accordance with the embodiments herein. On comparing the three sets of optical micrographs (FIG. 3), an evident change can be observed in the brightness contrast and surface morphology of the three gel types namely Leica 450 ms, ABET 8 s and ABET 80 s. For gels synthesized in Leica (FIG. 3, see (a)-(c)), the contrast was uniformly dark throughout the gel matrix with very sharp edges. However in Abet gels (FIG. 3, see (d)-(f) and (g)-(i)), with an increase in the exposure time to 8 s and 80 s (post application of ND filters), the edges lost their sharpness. Further, contrary to the Leica gels, where the polymerization seemed uniform along all directions, in Abet gels, the edges were underpolymerized compared to the central portion. This was evident in the lighter edge contrast due to lower crosslinking density caused by significant light attenuation at the photomask feature edges (FIG. 3); the extent of attenuation varies with the photomask design, the size, shape and distance between mask features. This variation in contrast can also be attributed to the difference of UV light sources. It is to be noted that Abet solar simulator is a flood illumination system where the UV light is uniformly irradiated over a 4 inch circular area (12566 inch²). Synthesis of gels in Abet is a one shot process where thousands of gels can be made in a single operation. The photomask is also kept in conformal contact with the top of the glass substrate of the gel device which faces the UV rays (FIG. 2). Contrary to the above, synthesis of gels in Leica DMi8 microscope can yield only one gel at a time since the UV beam emanating from the microscope illuminates a very small area of around 0.0004 inch². Moreover the photomask (consisting of a small circular single sheet of polymer pasted to a black tape with an orifice) in this case is integrated inside the optical filter cube in such a way that it lies underneath the emission filter. Such an arrangement allows the UV beam to pass through the photomask and illuminate a small area on the glass substrate of the gel device which is placed above at a distance from the filter cube on the sample stage. This narrow beam produces a very high UV power density in Leica (≈80 mW/cm²) compared to ABET (≈60 mW/cm²). This created gels with a high crosslinking density and a much smoother surface texture compared to the Abet ones which exhibited a highly corrugated surface. This effect became all the more evident in the bright field images of 80 s gels synthesized using ND1 filters (FIG. 3, see (g)-(i)). The change in surface texture is more clearly visible when the magnification was increased to 40× (FIG. 3, see (c), (f) and (i)).

FIG. 4 depicts gels synthesized with an exposure time (Tₑₓₚ) of 25 minutes (≈1500 s): wherein (a) depicts irregular shaped gels and wherein (b) and (c) depicts formation of droplets within the gel matrix, in accordance with the embodiments herein. As the UV exposure time was increased further to 25 minutes in presence of the ND2 filters (FIG. 4), irregular shaped gels started forming along with droplets due to N&G. The droplets comprised of the solvent rich aqueous phase with a high concentration of porogens (NaHCO₃ and PEG)). While the onset of N&G was clearly visible across all gel types synthesized with UV exposure times greater than 270 s, the porosity was found to be tunable/controllable for gel types synthesized with UV exposure time of upto about 660 s. Gel types synthesized with UV exposure time of 460 s were found to be tunable or controllable. At high UV exposure times (for eg: at exposure time equal to or greater than 660 s) the PEGDA phase separated from the prepolymer mixture dictated by N&G kinetics, resulting in the formation of a heterogenous microstructure with low pore interconnectivity. Shorter exposure time equal to or below 270 s assisted in the SD process allowing the bicontinuous structures to grow during polymerization without compromising mechanical integrity and microstructural homogeneity.

### Example 2

Hydrogels with Higher Exposure Time

The feasibility of gel synthesis at very high exposure time scales were studied using the same methodology, as in Example 1, where the effect of higher exposure times (10 minutes, 15 minutes, 17.5 minutes, 20 minutes, 25 minutes and 30 minutes (consisting of consecutive UV exposure of 10 and 20 minutes)) on the polymerization process was tested out on different prepolymer mixture formulations.

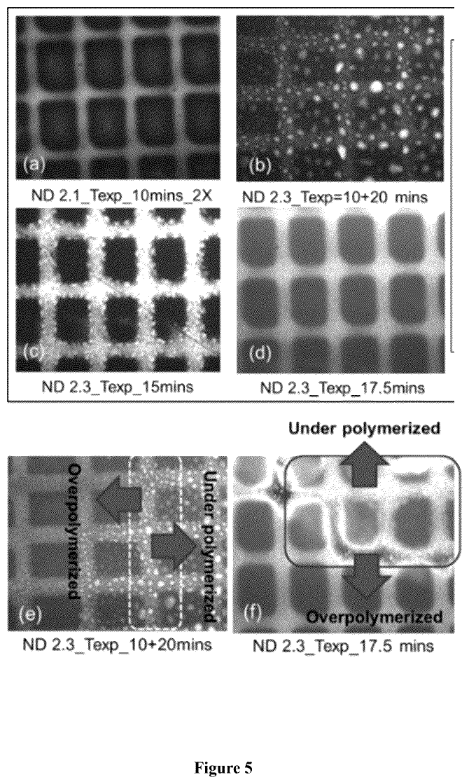

Prepolymer solutions (Non-working examples) having high ethylene-glycol based monomer (PEGDA) and photoinitiator concentrations along with a commensurate decrease in the porogen concentrations were prepared (Table 2.1) and subjected to UV curing in presence of higher OD ND filters (like ND 2, ND 2.1 & ND 2.3); the PEGDA concentration was increased from 19% w/v (as used in Example 1) to 28% w/v, 33.6% w/v and 39.2% w/v respectively along with a decrease in the NaHCO₃ concentration to 4% w/v from 5% w/v and PEG (Mol. Wt.=20 kDa) concentration to 3% w/v from 5% w/v respectively. The prepolymer solutions having 28% w/v PEGDA was the only one that showed phase stability before UV curing, while in all others, the porogens phase separated instantly after mixing. Polymerization was observed for this prepolymer solution, however, even after 25 mins of UV exposure with ND2 filters, patches of irregular shaped gels were formed. Lower exposure time of 23 minutes showed no appreciable difference in the polymerization kinetics, with the formation of patches of overpolymerized and underpolymerized gels in the gel device (FIG. 5). Similar observations were made for 10 min, 15 min and 17.5 minutes of UV exposure.

FIG. 5 depicts optical images of irregular shaped hydrogel microparticles synthesized with stacked neutral density filters (ND) and varying exposure time (Tₑₓₚ) at 10, 30, 15 and 17.5 minutes, wherein (a) depicts image of irregular shaped hydrogel microparticles synthesized with neutral density filters ND 2.1 at Tₑₓₚ (10 mins); (b) depicts image of irregular shaped hydrogel microparticles synthesized with neutral density filters ND 2.3 at Tₑₓₚ (30 mins); (c) depicts image of irregular shaped hydrogel microparticles synthesized with neutral density filters ND 2.3 at Tₑₓₚ (15 mins); (d) depicts image of irregular shaped hydrogel microparticles synthesized with neutral density filters ND 2.3 at Tₑₓₚ (17.5 mins); (e) depicts image of irregular shaped hydrogel microparticles synthesized with neutral density filters ND 2.3 at Tₑₓₚ (30 mins); and (f) depicts image of irregular shaped hydrogel microparticles synthesized with neutral density filters ND 2.3 at Tₑₓₚ (17.5 mins); Images taken at 2× magnification in the phase contrast mode, in accordance with the embodiments herein.

However, with a decrease in PI concentration to 0.432% w/v from 0.864% w/v and keeping the ethylene-glycol based monomer (PEGDA) and porogen (NaHCO₃ and PEG) concentration unaltered, 440 s and 660 s gels could be synthesized using ND1.8 and ND2 filters. Table 2.1 and Table 2.2 depicts the details of these non-working examples (NWE).

TABLE 2.1
Prepolymer solution used for Non-working examples:

| | | | | Ratio | | |
|---|---|---|---|---|---|---|
| | Ethylene | | | ethylene | | |
| | glycol- | | | glycol-based | | |
| | based | | | monomer | PBS | |
| | monomer | Photoinitiator | Porogen | and the | Buffer | |
| Description | (% w/v) | (% w/v) | (% w/v) | porogen | (% w/v) | Total (% w/v) |
| NWE1 | 28 | 0.86 | 10 | 2.8 | 61.14 | 100 |
| NWE2 | 33.6 | 0.43 | 7 | 4.8 | 58.97 | 100 |
| NWE3 | 39.2 | 0.43 | 7 | 5.6 | 53.37 | 100 |
| NWE4 | 33.6 | 0.86 | 7 | 4.8 | 58.54 | 100 |
| NWE5 | 39.2 | 0.86 | 7 | 5.6 | 52.94 | 100 |

TABLE 2.2
List of Non-working Examples.

| | | Light | Power | | |
|---|---|---|---|---|---|
| | ND Filter | Intensity | Density | UV Exposure | |
| Description | configurations | (mW) | (mW/cm2) | time (s) (Tₘ₎ | Observation |
| NWE1 | ND 2 | 0.14 | 0.18 | 10 mins | Patches of |
| NWE2 | ND 2.1 | 0.13 | 0.17 | 25 mins | over |
| NWE3 | ND 2.3 | 0.10 | 0.13 | 15 mins | polymerized |
| NWE4 | ND 2.3 | 0.10 | 0.13 | 17.5 mins | and under |
| NWE5 | ND 2.3 | 0.10 | 0.13 | 30 mins | polymerized |
| | | | | (consisting of | gels were |
| | | | | consecutive | observed. |
| | | | | shots of 10 and | |
| | | | | 20 mins) | |

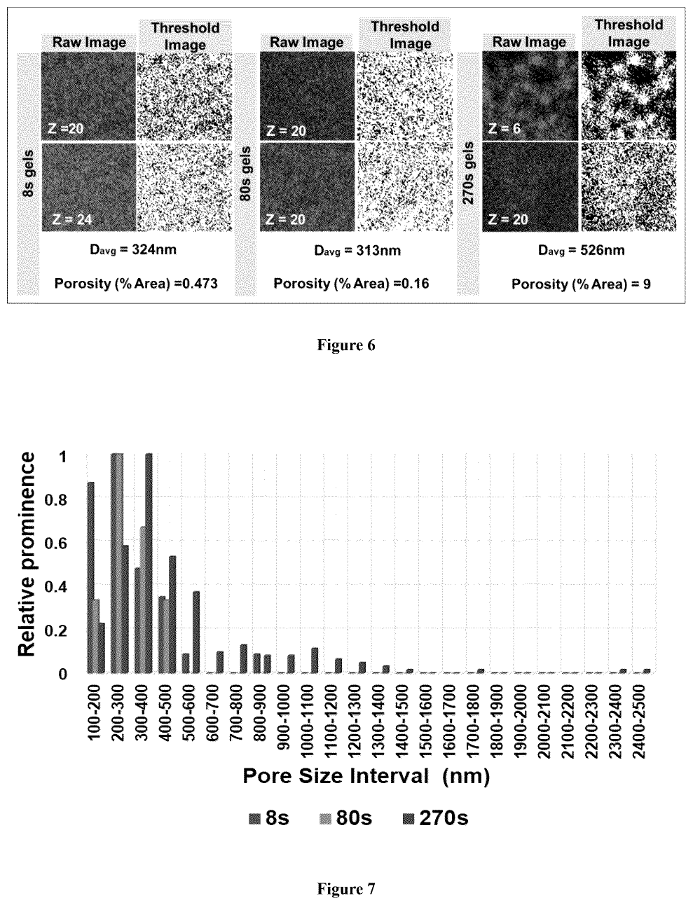

The confocal images in FIG. 6 showed an increase in the average pore diameter with increasing UV exposure time. Although, the average pore size of both 8 and 80 s gels were similar (≈310-320 nm), there was a sudden leap to 526 nm for 270 s gels. However, the evolution of pore size was more clearly visible on comparing the pore size distribution (FIG. 7). Among 8 s, 80 s and 270 s gels, the 80 s gels had the narrowest pore size distribution with almost 100% of the pores having diameter lying within the 100-500 nm range, followed by 8 s gels with a much broader profile within 100-900 nm. The 270 s gels had the broadest pore size distribution profile with 25-30% of pores with diameter >1 micron. When viewed within the interval 100-200 nm, 8 s gels had the maximum prominence followed by 80 s and 270 s. With increasing range (200-300 nm), the prominence of both 8 s and 80 s gels become comparable indicating an increase in the size of binodal structures. Again when viewed within the interval 300-400 nm, 270 s gels showed the maximum prominence indicating the coarsening of the binodal structures. 270 s gels also showed higher prominence for pores within the range 700-1500 nm.

### Scanning Electron Microscopy (SEM):

### Gel Lyophilization as a Preparatory Step for SEM Imaging:

Gels were washed with deionized (DI) water and vortexed at 1200 rpm for 2 min, followed by centrifugation at 2000 rpm for 10 s. The supernatant was discarded and the pellet was washed thrice with deionized (DI) water to ensure that no dissolved salts were present in the gel matrix. Use of PBS or any other buffer should be avoided for washing. 30 mL of liquid N₂ was poured in a 100 mL glass beaker and 200 μL of the gel dispersions were dispensed in the liquid N₂ using 200 μL tips cut with scissors to accommodate greater number of gel beads. Each droplet contained around 50-60 gels. Once the droplets froze in N₂, the excess N₂ was allowed to evaporate to avoid spillover, after which the frozen droplets were transferred into 30 mL glass bottle using a spatula and a glass/metal funnel. Some N₂ was left in the bottle so that the beads do not melt (liquid N₂ can be added in the bottle beforehand as a precooling step. The bottle was partially closed with a rubber cap and kept in the lyophilizer for 24 h; the condenser temperature was maintained at −50° C. and pressure at 100 m Torr. After 24 h, the bottle was taken out after the pressure was released, and the cap was sealed immediately to ensure no moisture got trapped inside the bottle. The cap was untouched for 30 min to avoid collection of moisture in the gels, after which the freeze-dried gels was further used for SEM characterization.

### Sem Imaging:

The lyophilized samples were taken out with the help of tweezers and placed on carbon tapes pasted on copper stubs. The grids were kept in vacuum desiccator for 8-10 h before imaging, after which they were coated with gold in a sputter coater (Quorum 150R); the coating was done for 50 s at a vacuum of 0.01 mBar. Gels were imaged at 500, 1000, 5000 and 10000× in ULTRA55 FE-SEM, KARL ZEISS, the operating voltage was kept within 3-5 kV. Image analysis was performed in ImageJ on images acquired at 10000× magnification. The porosity (%) is the area in percentage estimated from ImageJ analysis (https://www.academia.edu/24152953/Porosity_Analysis_Procedure_with_ImageJ; https://imagej.net/ij/docs/guide/146-30.html#toc-Section-30).

### Results

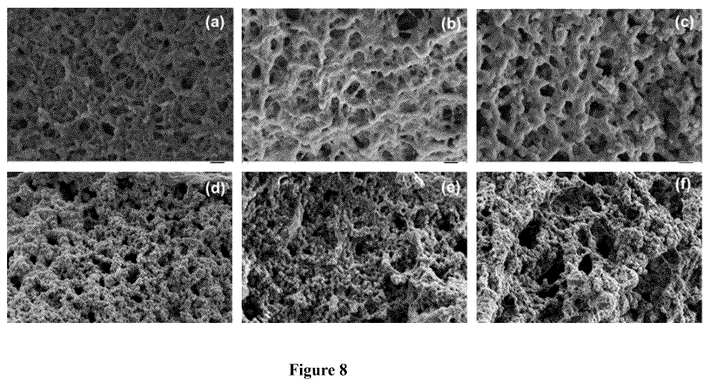

FIG. 8 shows the SEM micrographs of 8 s (a)-(b), 80 s (c), 270 s (d), 480 s (e) and 660 s (f) gels. From the micrographs, it is clearly evident that all the gel types were macroporous with pores having diameters ranging within 150-500 nm. However, qualitatively, it can be inferred that the microstructure has evolved from 8 s to 80 s gels in agreement with the confocal data as shown in FIGS. 6 and 7. The emergence of pits (i.e. large voids) in the gels indicate the onset of nucleation and growth (N&G). As the exposure time increases further to 480 and 660 s, the phase separated PEG, NaHCO₃ and water molecules create droplets that coalesce with time to create bigger droplets by a process known as Ostwald Ripening, thereby leading to isolated micron sized voids scattered throughout the gel matrix. This imparts a high degree of heterogeneity of the polymer network.

TABLE 3
Pore size and porosity estimation of hydrogels from FE-SEM microscopy,
for Working examples 1 to 4 and Non working example 6.

| | Gel Type | Average | Average | |
|---|---|---|---|---|
| | (based on | pore area | Pore Size | Porosity |
| Description | Tₘ₎ | (nm²) | (nm) | (%) |
| WE1 | 8 s | 29711.13 | 194 | 19.468 |
| WE2 | 80 s | 59766.35 | 275 | 31.226 |
| WE3 | 270 s | 97978.73 | 353 | 25.17 |
| WE4 | 480 s | 169282.2 | 464 | 33.655 |
| NWE6 | 660 s | 3099.717 | 63 | 8.74 |

### Significance Analysis:

To ascertain the statistical significance between the pore size due to different exposure times, one-way ANOVA along with Tukey's Post hoc test is performed. Here, the different exposure times, namely 8 s, 80 s, 270 s, 480 s, and 660 s are considered different treatments. The influence of these treatments on pore size is evaluated. The p-value corresponding to the F-statistic of one-way ANOVA is lower than 0.05, suggesting that one or more treatments are significantly different. The post-hoc tests would likely identify which of the pairs of treatments are significantly different from each other.

### Results:

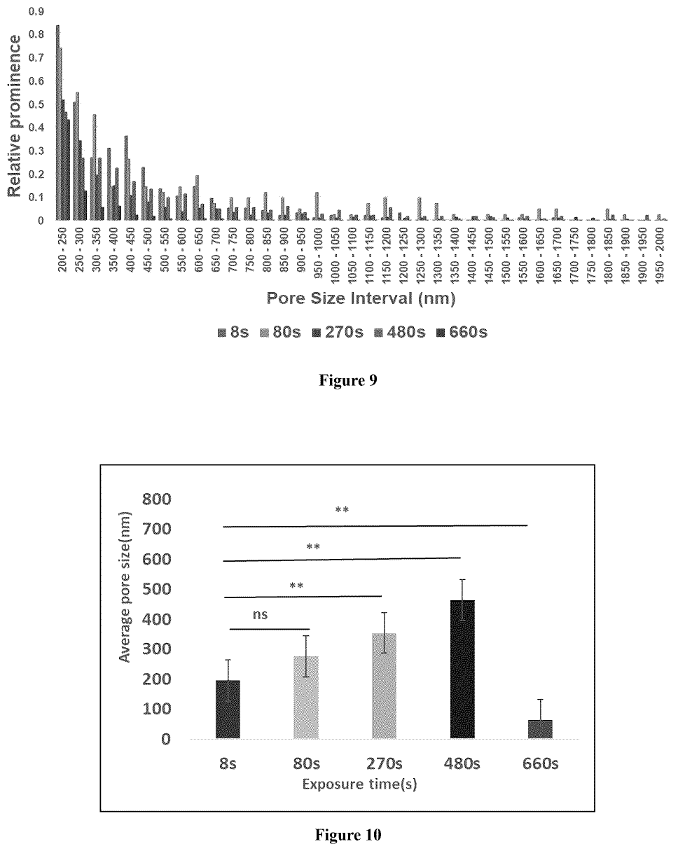

The SEM images showed significant differences between 8 s and 80 s, 270 s, 480 s and 660 s (p<0.01), as depicted in FIG. 10.

The average pore size was observed to drop at higher exposure time of 660 s. Hence, it may be inferred that at higher exposure time, the porosity of gels were not tunable.

### Example 3

### Preparation and Characterization of Macroporous Hydrogel Sensor

### Preparation of Macroporous Hydrogel Sensor

The hydrogels prepared in Example 1 were used to prepare the sensors. The gels were hexagonal in shape having lateral dimensions of 460-480 microns. The gels were post-synthesis-functionalized (PSF) by contacting with a capture antibody (CAb) (conc.=0.5 mg/mL, capture agent) as per an in-house developed protocol to obtain the macroporous hydrogel sensor. Briefly, the gels were incubated in CAb solution mixed with thiolated-PEG linker. The thiolated-PEG has one arm with thiol (S—H) group which binds to the C═C of PEGDA and the other arm having N-hydroxysuccinimide (NHS)-ester which reacts with the primary amines of capture antibody thus anchoring the antibody with the PEGDA matrix.

### Fluorescence Immunoassay for the Detection of Thyroid Stimulating Hormone (TSH)

Fluorescence immunoassay was performed with 8 s, 80 s, 270 s and 480 s hydrogel sensors as prepared herein above for the estimation of the LLOD for TSH (analyte). Assays were performed in triplicates in 96 black well plates. 8-10 sensors were added in each well followed by the addition of serum calibrators mixed with fluorescently labelled detection antibodies (Dab, detecting agents). The gels were incubated for 20 min with fixed volumes of the analyte comprising of a mixture of calibrators and dye labelled detection antibody DAb (referred to as the sample mix) (conc.=1 μg/mL) prepared as per in-house developed protocol. Briefly, the measured volumes of detection antibody (Dab) conjugated to a fluorescent dye was mixed with TSH calibrators in fixed proportions. The TSH calibrators were made from stripped human serum and then spiked with calibrated amounts of TSH hormone. Each calibrator concentration represents the concentration of the TSH hormone in the stripped serum samples. This was followed by rinsing and washing with buffer solution (PBS+0.1% Tween 20) after which the gels were imaged in Leica DMi8 microscope in the fluorescence mode at an excitation wavelength of 635 nm. For 8 and 80 s gels, four different calibrators namely A, E, G and I were used, each having different concentration of the analyte, TSH 0.05 μIU/mL for A, 1.83 μIU/mL for E, 7.74 μIU/mL for G, and 26.59 μIU/mL for I. The acquired signal was plotted against the antigen (analyte) concentration to generate a calibration curve from which the LLOD was estimated. For 270 and 480 s gels, 5 different calibrators were used (viz., A, C, E, G, and I cals), the C calibrator TSH concentration was 0.55 μIU/mL. One set of gels of each type were run without any calibrators (stripped serum denoted as A0). To estimate the background signal one set of gel sensors without Cab (used as negative controls) were incubated with only the Dab mixed in PBST buffer. Lowest antigen concentration was estimated from the calibration curve according to the relation.

### Results

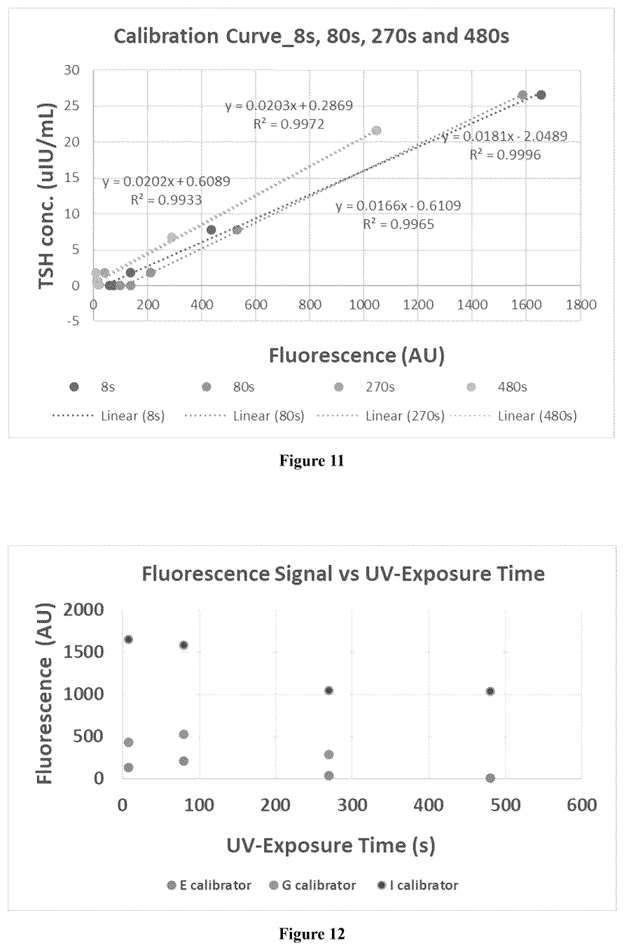

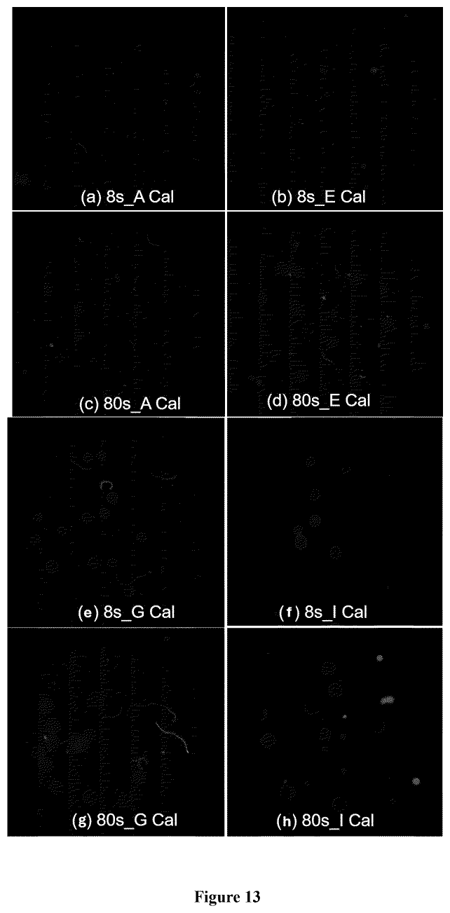

Lowest Limit of Detection was estimated from the five point calibration curve from TSH assay (FIG. 9) using the formula:

$$y = m ( {FL}_{A calibrator} + 3 σ ) + c$$

where, m is the slope of the linear fitted calibration curve (FIG. 11), FL_{A-calibrator} is the fluorescence signal at the lowest antigen concentration i.e. for A calibrator, σ is the standard deviation of the inter-well fluorescence (Table 4 & 5) for A calibrator and c is the y-intercept of the fitted calibration curve (FIG. 11). There was a dip in the LLOD in 80 s gels vis a vis 8 s ones. The LLOD of 80 s gels was estimated to be around ≈0.6 μIU/mL. The average fluorescent signal for 80 s gels was higher at lower cals (A and E) compared to the 8 s ones. Similar increase in signal intensity was also observed for E and G calibrators, which exhibited very bright gel edges (FIGS. 12 & 13). FIG. 13 depicts fluorescence images (imaged in Leica DMi8 microscope) of 8 s and 80 s hydrogel microsensors undergoing TSH immunoassay with four calibrators (A, E, G and I), wherein (a) depicts 8 s gel (A calibrator) and (b) depicts 8 s gel (E calibrator); (c) depicts 80 s gel (A calibrator), (d) depicts 80 s gel (E calibrator); (e) depicts 8 s gel (G calibrator); (f) depicts 8 s (I calibrator); (g) depicts 80 s gel (G calibrator); and (h) depicts 80 s gel (I calibrator), in accordance with the embodiments herein.

Overall, the signal resolution was found to improve for 80 s gels, and were adjudged the best for TSH immunoassay. Among several factors that affect TSH assay sensitivity in hydrogels, the enhanced porosity was a very important one which led to improved washout of unbound CAb molecules leading to a dip in the background signal. Considering stokes diameter of ≈10-11 nm for IgG (MW=150 kDa) and ≈4-6 nm for TSH molecule (MW=28 kDa), the size of the immunocomplex was expected to vary within the range ≈24-28 nm. 80 s gels with average pore size falling within the range of ≈275-313 nm was roughly ten times the size of the immunocomplex and adjudged to be the most promising candidate for developing future TSH and/or IgG based immunoassays.

Table 4 depicts TSH immunoassay data for 8 s gels showing interwell and intrawell average signal intensity standard deviation, % CV and SEM for A, E G and I calibrators containing 0.05, 1.83, 7.74, and 26.59 uIU/mL TSH concentration; A0 calibrator corresponds to stripped serum sample which does not contain TSH.

TABLE 4
8 s gels

| Calibrator | | A0 | | | A | | | E | | | G | | | I | | |
|---|---|---|---|---|---|---|---|---|---|---|---|---|---|---|---|---|
| TSH Conc. (uIU/mL) | | 0.00 | | | 0.05 | | | 1.83 | | | 7.74 | | | 26.59 | | |
| Intrawell | Average | 53 | 86 | 45 | 110 | 71 | 41 | 123 | 123 | 170 | 398 | 460 | 454 | 1687 | 1626 | 1652 |
| | Signal | | | | | | | | | | | | | | | |
| | SD | 29 | 18 | 19 | 12 | 18 | 13 | 55 | 55 | 46 | 88 | 150 | 229 | 174 | 155 | 202 |
| | % CV | 55 | 21 | 42 | 11 | 25 | 32 | 45 | 45 | 27 | 22 | 33 | 51 | 10 | 10 | 12 |
| | SEM | 21 | 10 | 13 | 9 | 10 | 9 | 32 | 32 | 21 | 51 | 87 | 162 | 87 | 78 | 90 |

| | | | | | | |
|---|---|---|---|---|---|---|
| Interwell | Average | 61 | 74 | 139 | 437 | 1655 |
| | Signal | | | | | |
| | SD | 22 | 35 | 27 | 34 | 31 |
| | % CV | 35 | 47 | 19 | 8 | 2 |
| | SEM | 10 | 16 | 10 | 15 | 10 |

Table 5 depicts TSH immunoassay data for 80 s gels showing interwell and intrawell average signal intensity standard deviation, % CV and SEM for A, E G and I calibrators containing 0.05, 1.83, 7.74, and 26.59 uIU/mL of TSH concentration; A0 calibrator corresponds to stripped serum sample which does not contain TSH.

TABLE 5
80 s gels

| Calibrator | | A0 | | | A | | | E | | | G | | | I | | |
|---|---|---|---|---|---|---|---|---|---|---|---|---|---|---|---|---|
| TSH Conc. (uIU/mL) | | 0.00 | | | 0.05 | | | 1.83 | | | 7.74 | | | 26.59 | | |
| Intrawell | Average | 102 | 105 | 92 | 137 | 134 | 139 | 245 | 218 | 177 | 508 | 469 | 622 | 1655 | 1650 | 1452 |
| | Signal | | | | | | | | | | | | | | | |
| | SD | 26 | 13 | 43 | 7 | 55 | 28 | 129 | 5 | 20 | 47 | 53 | 58 | 516 | 93 | 163 |
| | % CV | 25 | 12 | 46 | 5 | 41 | 20 | 12 | 2 | 11 | 9\| | 11 | 9 | 31 | 6 | 11 |
| | SEM | 15 | 7 | 21 | 4 | 211 | 16 | 12 | 3 | 9 | 21 | 27 | 29 | 231 | 42 | 82 |

| | | | | | | |
|---|---|---|---|---|---|---|
| Interwell | Average | 100 | 137 | 213 | 533 | 1586 |
| | Signal | | | | | |
| | SD | 7 | 3 | 34 | 80 | 116 |
| | % CV | 7 | 2 | 16 | 15 | 7 |
| | SEM | 3 | 1 | 12 | 28 | 39 |

### Advantages of the Present Disclosure

The present disclosure relates to a process for preparing macroporous hydrogels with the following advantages.
a) Contrary to conventional multistep methods for fabricating of macroporous gels the disclosed process is a single-step process where only bicontinuous macro domains are produced, and photo polymerization induced phase separation (PIPS) technique is used for pore creation.
b) The coarsening effect of pore enlargement with improved pore interconnectivity can be tuned simply by changing light intensity by applying ND Filters of known optical density singly or in combination to cut out UV irradiation.
c) Contrary to other semi crystalline polymers which generate a macroporous closed cellular structure with a fast-cooling rate undergoing thermal polymerization, PEGDA 700 being an amorphous oligomer, creates a macroporous open cellular structure with slow UV curing.
d) Photolithography helps in making the hydrogel micro sized replicas with desired shapes in a very short span of time and with a high degree of repeatability and throughput. This paves the way for large scale industrial production of hydrogel sensors.
e) Macroporous hydrogel sensors are synthesized with mean pore size 100 to 500 nm or 200-300 nm and lateral dimensions of 300-500 microns.
f) The macroporous hydrogel sensors with fine-tuned microstructure and desired shapes enables their integration in microfluidic POCT Devices for multiplexed immunoassays.

I/We claimed:

1. A process for preparing a macroporous hydrogel, the process comprising:
a. exposing a prepolymer solution comprising an ethylene glycol-based monomer, a photoinitiator, and at least one porogen, to UV radiation for a duration in the range of 0.1 s to 600 s, to form a hydrogel, wherein power density of the UV radiation is in the range of 0.1 to 70 mW/cm²; and
b. washing the hydrogel, to obtain the macroporous hydrogel.

2. The process as claimed in claim 1, wherein exposing the prepolymer solution to UV radiation induces spinodal decomposition in the prepolymer solution.

3. The process as claimed in claim 1, wherein the UV radiation passes through a photomask and a neutral density filter, wherein the neutral density filter has optical density in a range of 0.1 to 1.8.

4. The process as claimed in claim 1, wherein the ethylene glycol-based monomer and the porogen are in a weight ratio range of 1:50 to 50:1.

5. The process as claimed in claim 1, wherein the ethylene glycol-based monomer is selected from poly(ethylene glycol) di-acrylate (PEGDA), poly(ethylene glycol) methacrylate (PEGMA), poly(ethylene glycol) dimethacrylate (PEGDMA), PEG-norborene, or combinations thereof; and the ethylene glycol-based monomer is in an amount in a range of 10 to 40% w/v with respect to the prepolymer solution.

6. The process as claimed in claim 4, wherein the ethylene glycol-based monomer is PEGDA having an average molecular weight in the range of 250 to 6000 Da.

7. The process as claimed in claim 1, wherein the photoinitiator is selected from 2-hydroxy-2-methyl-propiophenone, lithium phenyl-2,4,6-trimethylbenzoylphosphinate, 2-hydroxy-4′-(2-hydroxyethoxy)-2-methylpropiophenone, or combinations thereof; and the photoinitiator is in an amount in the range of 0.1 to 2% w/v with respect to the prepolymer solution.

8. The process as claimed in claim 1, wherein the porogen is selected from a water-soluble polymer, an inorganic compound, or combination thereof.

9. The process as claimed in claim 8, wherein the water-soluble polymer is selected from PEG, gelatin, agarose, alginate, or combinations thereof; and the inorganic compound is selected from sodium bicarbonate, silica, calcite, or combinations thereof.

10. The process as claimed in claim 8, wherein the water-soluble polymer is in an amount in a range of 2 to 6% w/v with respect to the prepolymer solution; or wherein the inorganic compound which is in an amount in a range of 2 to 6% w/v with respect to the prepolymer solution, or wherein the porogen is in an amount in a range of 1 to 10% w/v with respect to the prepolymer solution.

11. The process as claimed in claim 1, wherein the prepolymer solution comprises an aqueous buffer selected from phosphate buffered saline (PBS), tris-buffered saline (TBS), or combinations thereof.

12. The process as claimed in claim 1, wherein the photomask is selected from laser-plotted polyester-based photomask, glass based chrome films, or quartz-based chrome films.

13. The process as claimed in claim 1, wherein the macroporous hydrogel comprises pores having an average pore size in a range of 100 to 500 nm; and porosity in a range of 10 to 40%.

14. The process as claimed in claim 1, further comprising contacting the macroporous hydrogel with at least one capture agent and incubating to enable functionalization of the capture agent on the macroporous hydrogel to obtain a macroporous hydrogel sensor, wherein the capture agent is selected from an antibody, an oligonucleotide, an enzyme, an antigen, or combinations thereof.

15. The process as claimed in claim 1, further comprising lyophilizing said macroporous hydrogel to obtain a lyophilized macroporous hydrogel.

16. A macroporous hydrogel having an average pore size in a range of 100 to 500 nm; and porosity in a range of 10 to 40%, obtained by the process as claimed in claim 1.

17. A macroporous hydrogel sensor obtained by the process as claimed in claim 14.

18. A method for detecting an analyte in a sample, said method comprising contacting the macroporous hydrogel sensor as claimed in claim 17 and the sample, and detecting the presence or absence of the analyte in the sample.

19. The method as claimed in claim 18, wherein the analyte is selected from a hormone, a immunoglobulin, or an antigenic protein.

20. A kit comprising the macroporous hydrogel obtained by a method as claimed in claim 1, and an instruction manual.
